(19) United States
(12) Patent Application Publication
(10) Pub. No.: US 2010/0112617 A1
(43) Pub. Date: May 6, 2010

(54) Evaluating RTK Target Drugs

(75) Inventor: Darren W. Davis, Houston, TX (US)

Correspondence Address: BAKER & MCKENZIE LLP, Pennzoil Place, South Tower, 711 Louisiana, Suite 3400, HOUSTON, TX, 77002-2716, US

(73) Assignee: Apocell, Inc, Houston, TX (US)

(21) Appl. No.: 12/532,396

(22) PCT Filed: Mar. 7, 2008
(86) PCT No.: PCT/US08/56208

§ 371 Date: Jan. 11, 2010

Related U.S. Application Data
(60) Provisional application No. 60/895,981, filed on Mar. 20, 2007.

(51) Int. Cl. C12Q 1/48; G06F 17/30; C12Q 1/02

(52) U.S. Cl. 435/15; 707/802; 435/29; 707/748; 707/E17.044

(57) ABSTRACT

Methods of evaluating receptor tyrosine kinase drug efficacy are demonstrated. The methods generally relate to evaluation methods using phospho-RTK over total RTK ratio (pRTK/tRTK). An algorithm is provided that allows the user to combine the pRTK/tRTK ratios from several kinase together with other kinds of measurements to obtain a PDX value that is indicative of drug efficacy.

### PRIOR RELATED APPLICATIONS

This application claims priority to U.S. Provisional Application 60/895,981 filed Mar. 20, 2007, incorporated herein by reference in its entirety.

### FEDERALLY SPONSORED RESEARCH STATEMENT

The present invention may have been developed with funds from the United States Government. Therefore, the United States Government may have certain rights in the invention.

### REFERENCE TO MICROFICHE APPENDIX

Not applicable.

### FIELD OF THE INVENTION

The invention relates to measurement of receptor tyrosine kinase and measurements thereof that relate to assessing the efficacy of drugs that target RTKs.

### BACKGROUND OF THE INVENTION

Receptor tyrosine kinases (RTKs) act as signals initiating a variety of kinase cascades and are involved in diverse processes from epithelial growth to apoptosis. RTKs are thus very important targets for drug development.

There are over 1500 receptor tyrosine kinases (RTKs) available in the GENBANK® database (www.ncbi.nlm.nih.gov/entrez). One subset of RTKs useful for slowing or inhibiting cancer growth is involved in angiogenesis. By inhibiting RTKs that induce angiogenesis, blood flow to tumors can be restricted and their growth stopped. RTKs involved in angiogenesis include epidermal growth factor receptor (EGFR), platelet derived growth factor receptor (PDGFR), vascular endothelial growth factor receptor (VEGF-R1 and 2), TIE receptor tyrosine kinase (tyrosine kinase with immunoglobulin-like and EGF-like domains), and epithelial cell receptor protein-tyrosine kinases (EPHA and EPHB) as well as non-receptor RTKs PYK2 and c-SRC, among others.

Several RTK inhibitors (RTKIs) have already been approved by the FDA for cancer treatment and many more are in various stages of development. For example, Cetuximab (ERBITUX®) is an antibody inhibitor of EGFR, and has proven useful in treating squamous cell carcinoma and colorectal cancer. Trastuzumab (HERCEPTIN®) is a humanized monoclonal antibody that acts on the HER2/neu (erbB2) receptor. Trastuzumab's principal use is as an anti-cancer therapy in breast cancer in patients whose tumors overexpress erbB2. Gefitinib and erlotinib (TARCEVA® or OSI-774, OSI PHARMACEUTICALS™, Uniondale, N.Y.) are small molecule inhibitors of the EGFR tyrosine kinase. Other RTKIs include dasatinib, erlotinib, imatinib, lapatinib, nilotinib, sorafenib, sunitinib, and vandetanib (ZD6474), among others.

A variety of RTKIs are available, but methods of predicting RTKI clinical efficacy based on easily measured biochemical endpoints are required to improve RTKI screening. Inhibition of RTK phosphorylation (pRTK) has been used to monitor RTKI activity. However, several RTKI clinical trials failed to correlate pRTK inhibition with clinical response. Thus, pharmaceutical companies continue to look for methods of predicting clinical efficacy based on easily measured biochemical parameters.

### SUMMARY OF THE INVENTION

The invention is based on the discovery that RTKI's can inhibit RTK phosphorylation and simultaneously change RTK expression levels. This explains, at least in part, the failure of phospho-RTK to correlate with clinical efficacy, and underscores a need to measure both phospho-RTK as well as total-RTK levels to obtain an accurate picture of the state of RTK inhibition. Thus, the invention is directed to the predictive ability of the pRTK/tRTK ratio, methods of measuring the pRTK/tRTK ratio, uses of the pRTK/tRTK ratio to diagnose and treat patients, and algorithms related thereto.

The pRTK/tRTK ratio can be used in high throughput screening to predict the clinical efficacy of a test drug. The method can be applied to a variety of cell types, such as cancer cells, and thus data collected simultaneously about a variety of cancers. Measurement and assessment of pRTK/tRTK can be used to create databases that predict clinical response of various cell types with a variety of drugs. pRTK/tRTK can be measured in various cancer cell lines, endothelial cells, epidermal cells, and tissue samples.

The pRTK/tRTK ratio can also be used to predict and monitor an individual patient's response to a particular drug. It provides a biomarker for monitoring, dosing, scheduling, and frequency of administration. An initial pRTK/tRTK ratio obtained at diagnosis predicts the clinical response of patients and determines initial treatment options. pRTK/tRTK ratio during and post-treatment is used to monitor ongoing treatments, identify drug resistance, and determine when new treatment options should be initiated.

In addition to employing the simple two parameter pRTK/tRTK ratio as a measure of efficacy, we have developed an algorithm for collecting and assessing information from a plurality of biological markers, such as positive and negative tumor factors and/or a variety of pRTK/tRTK ratios. The algorithm identifies the likelihood of whether the patient will respond to therapy using the novel scoring system, identifies extent of molecular effect with clinical outcome or response information, e.g., partial response, stable disease, etc, and combines molecular and cellular effects with other imaging technologies, e.g., PET, MRI, CT to determine which RTKI will elicit a desired clinical response.

Methods are described for screening potential RTKIs by exposing cells to a test agent, measuring pRTK and tRTK levels, determining the pRKT/tRTK ratio. If the pRTK/tRTK ratio decreases in a dose dependent manner, then the RTKI inhibits pRTK activity.

Methods of monitoring efficacy of an RTKI treatment are also disclosed. A cell having RTK activity is taken from a patient with an RTK mediated condition, pRTK is measured, tRTK is measured, and the pRTK/tRTK ratio determined. If the pRTK/tRTK levels are low, the RTKI treatment is effective.

### BRIEF DESCRIPTION OF THE DRAWINGS

FIG. 1. pKDR/tKDR Correlation with Clinical Response. pKDR and tKDR were measured from patient tumor biopsies and compared against known clinical response when treated with TARCEVA® (erlotinib) plus or minus AVASTIN® (bevacizumab). CR=complete response, SD=stable disease, PD=progressive disease. The pKDR/tKDR ratio correlates directly with clinical response in head (H) and neck (N) cancer patients treated with erlotinib±bevacizumab.

FIG. 2. Response Prediction Curve. Using the pKDR/tKDR ratio for head (H) and neck (N) patients treated with bevacizumab±erlotinib derived from FIG. 1 (solid line) and extrapolated therefrom (dotted line). CR=complete response, SD=stable disease, PD=progressive disease. Patient response to bevacizumab±erlotinib treatment could be predicted for patients X and Y.

FIG. 3. PDX Value versus Clinical Outcome. In general, a low the PDX value predicts a good response to treatment and continued therapy, whereas a high PDX value suggests a new treatment should be initiated. CR=complete response, SD=stable disease, PD=progressive disease.

### DETAILED DESCRIPTION OF THE INVENTION

The present invention is exemplified with respect to EGFR and AEE78, PDGFR-beta and SU11248, and KDR and erlotinib and bevacizumab. However, the method is generally applicable to RTKs and RTKIs. Further, we exemplified the invention with cancer drugs, but any RTKI inhibitor can be employed in the invention and any RTK-mediated disease can thus be evaluated by the methods described herein.

Phospho-RTK and total-RTK levels are measured independently or concurrently using a variety of technology platforms. Phospho-RTK can be measured by anti-pRTK antibody, radiolabeling, or fragmentation and mass spectroscopy. Total RTK levels can be measured by immunoassay, radiolabeling or fragmentation and mass spectroscopy. Immunodetection can be performed through either a solution phase or solid phase assay and in any format including, for example, dot blot, dip stick, ELISA, Western, or flow cytometry. However, in a preferred method the immunodetection is automatically quantitated using a Laser Scanning Cytometry (LSC) platform and software. LSC may also be used to detect mRNA levels using fluorescent in situ hybridization (FISH).

Terminal Deoxynucleotidyl Transferase Mediated dUTP Nick End Labeling (TUNEL) is a common method for detecting DNA fragmentation that results from apoptotic signaling cascades. The assay relies on the presence of nicks in the DNA which can be identified by terminal deoxynucleotidyl transferase, an enzyme that will catalyze the addition of dUTPs that are secondarily labeled with a marker. The TUNEL assay was originally described by Garvrieli, Sherman, and Ben-Sasson, incorporated herein by reference. Additionally, TUNEL specificity for apoptosis has been increased using the methods of Negoescu, et al. (Negoescu, 1996; Negoescu, 1998), incorporated herein by reference.

As used herein “tumor promoting factor” or “TPF” is a measurement that increases during tumor activity. Some examples of TPFs are proliferation, phosphorylation of RTKs (including VEGFR, PDGFR, EGFR, PYK2, and SRC), expression of growth factors (including VEGF, PDGF, and EGF) and the like. Thus the TPF for a pRTK/tRTK is the ratio of pRTK/tRTK before and after treatment.

As used herein, “tumor suppressing factor” or “TSF” is a measurement that increases due to tumor inhibition or death, i.e. apoptosis, tumor remission, and the like. Ratios normalize results and thus provide an easy to interpret change in signal. In one embodiment, the TSF for a TUNEL score is the ratio of TUNEL score before and after treatment.

As used herein, “PDX” or “PharmacoDynamic eXpression” value means:

PDX=[(100+TPF1)₁+(100+TPF2)₂+ . . . (100+TPFN)ₙ+(100−TSF1)₁+(100−TSF2)₂+ . . . (100−TSFM)ₘ]/(n+m)

As used herein, “RTK-mediated disease” is a disease mediated in large part by one or more RTKs and is thus responsive to drugs that target such RTKs, including hyperprolifative diseases, inflammatory responses, and the like.

### EXAMPLE 1

### Materials and Methods

TABLE 1
Cell strains and antibodies

| Product | Description | Source |
|---|---|---|
| HY-29 | epithelial cells from colorectal | ATCC: HTB-38 |
| cells | adenocarcinoma of the colon | |
| GIST | gastrointestinal stromal tumor | |
| HUVEC | human umbilical cord endothelial | |
| | cells | |
| P-EGFR- | anti-phospho-EGFR antibody | CELL SIGNALING ®, |
| Y1173 | | Beverly, MA |

### EXAMPLE 2

### RTK Response to RTKI

We sought to test RTK response to RTKI's and thus measured both phospho-RTK and total RTK using a known RTKI challenge reagent. AEE788 (NOVARTIS®) is an oral multiple-receptor tyrosine kinase inhibitor of EGFR, human epidermal growth factor receptor 2 (HER-2), and vascular endothelial growth factor receptor (VEGFR). Thus, we measured EGFR response to AEE788 using antibody quantitation of the two parameters.

HT-29 colon cancer cells were treated with increasing doses of AEE788, (0, 4, 20, 100, and 1000 nM) for 4 hours in serum-free media followed by EGF stimulation at 100 ng/ml for 5 minutes. Cells were then fixed and stained with anti-phospho-EGFR (P-EGFR-Y1173) antibody followed by a secondary antibody conjugated with a fluorescent probe. The mean fluorescence index (MFI) of pEGFR was quantified by laser scanning cytometry (LSC) nm and the results are shown in Table 2.

TABLE 2
Treatment of HT-29 colon cancer cells (0% FCS) with AEE788

| | EGF | | | |
|---|---|---|---|---|
| AEE788 | (100 ng/ml | | | pEGFR/ |
| (nM) for 4 h | for 5 min) | pEGFR* | tEGFR* | tEGFR* |
| 0 | No | 1.00 | 1.00 | 1.00 |
| 0 | yes | 2.18 | 1.77 | 1.23 |
| 4 | yes | 1.36 | 2.11 | 0.65 |
| 20 | yes | 0.97 | 4.61 | 0.21 |
| 100 | yes | 0.70 | 4.37 | 0.16 |
| 1000 | yes | 0.52 | 3.53 | 0.15 |
| AG1478 (10 mM) | yes | 0.60 | 2.12 | 0.29 |

*fold change in level

As expected, AEE788 inhibited EGF-induced phosphorylation of its receptor (pEGFR) in a dose-dependent manner. However, to our surprise, AEE788 also up-regulated total EGFR levels. This suggested that in order to effectively assess inhibitor activity, the pEGFR/tEGFR ratio should be monitored, rather than the pEGFR alone.

### EXAMPLE 2

### RTK Response to RTKI is General

We next sought to ensure that the RTKI effect on total RTK level was a general phenomenon, and not an isolated effect specific to AEE788 and EGFR. Thus, we repeated the experiment using additional RTKs and RTKIs. We used a known RTKI challenge reagent SU11248, which blocks phosphorylation of several kinases, including VEGFR2 (KDR), stem cell tyrosine kinase receptor (KIT), platelet-derived growth factor receptor (PDGFR), and fms-related tyrosine kinase 3 (FLT3). We found that SU11248 inhibits the phosphorylation of PDGFR-β, and that the pPDGFR-β/tPDGFR-β ratio decreased in a dose-dependent manner (Table 3) in a GIST and HUVEC cells when treated with SU11248. A similar response was seen with the RTK and erlotinib and bevacizumab combination (data not shown).

TABLE 3
Dose dependent pPDGFR-β/tPDGFR-β with SU11248

| | | GIST | HUVEC |
|---|---|---|---|
| SU11248 | PDGF | pPDGFR-β/ | pPDGFR-β/ |
| (μM) | (50 ng/ml) 10 min | tPDGFR-β* | tPDGFR-β* |
| 0 | No | 1.00 ± 0.00 | 1.00 ± 0.00 |
| 0 | Yes | 1.80 ± 0.08 | 4.99 ± 1.81 |
| 0.01 | Yes | 0.97 ± 0.05 | 2.42 ± 0.36 |
| 0.1 | Yes | 0.00 ± 0.59 | 1.48 ± 0.15 |
| 1 | Yes | 0.00 ± 0.08 | 1.40 ± 0.23 |

*fold pPDGFR-β/tPDGFR-β ratio change

### EXAMPLE 3

### Correlation with Clinical Outcome

Bevacizumab (AVASTIN®) is a monoclonal antibody that inhibits the activity of VEGF. It is used to treat colorectal, renal cell, ovarian, lung and breast cancers. It is used alone or in combination with 5-fluorouracil (5-FU), leucovorin, and oxaliplatin or irinotecan. Bevacizumab can also be combined with erlotinib (TARCEVA®) to increase efficacy. Erlotinib, similar to gefitinib, specifically targets EGFR tyrosine kinases. Erlotinib is effective against lung, pancreatic, adenocarcinoma and other cancers. Erlotinib is also effective for inhibition of JAK2V617F, a mutant JAK2 tyrosine kinase, found in most patients with polycythemia vera (PV) and other myeloproliferative disorders.

pKDR/tKDR ratios in baseline tumor biopsies were correlated with clinical response for treatment with bevacizumab. The MFI of total pKDR and KDR obtained from samples collected at baseline were used to calculate the pKDR/tKDR ratio. Student's T test was used to determine the p values between complete response (CR), stable disease (SD), and progressive disease (PD).

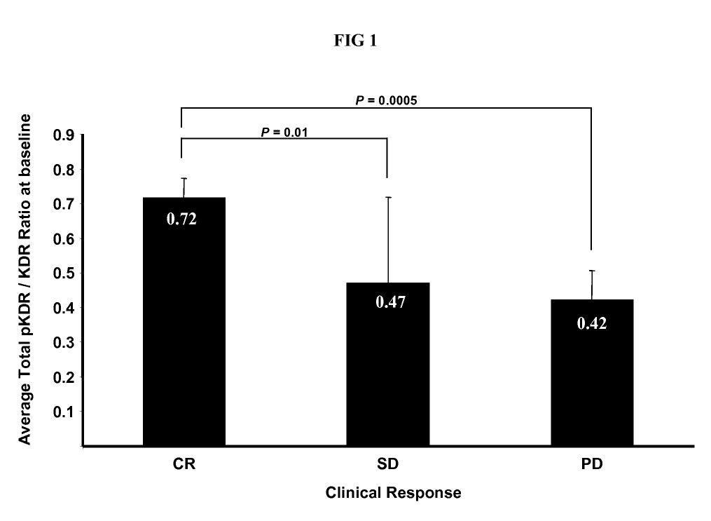

Our results showed that higher pKDR/tKDR ratios indicated better response to bevacizumab±erlotinib treatment for patient with head and neck cancers (FIG. 1). This data suggests that pKDR/tKDR ratio in tumor biopsies obtained at diagnosis can be used as a predictive marker to stratify patients for cancer treatment.

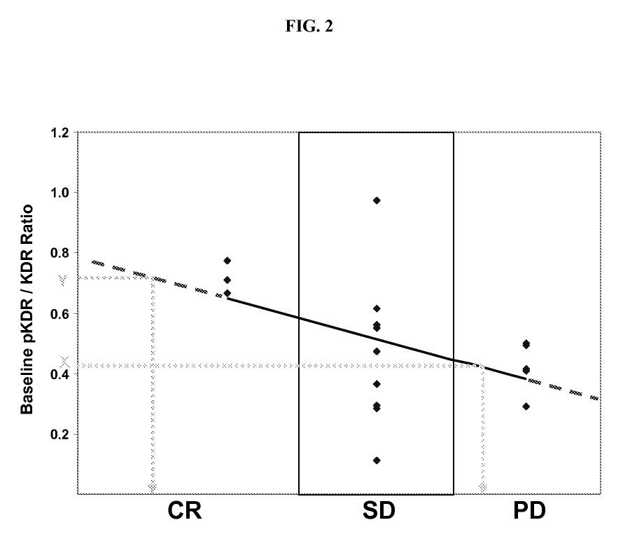

Next we provided a response prediction curve based on the pKDR/tKDR ratios in tumor biopsies from FIG. 1. The solid line in FIG. 2 was derived from the data obtain from FIG. 1, and the extrapolated data is the dotted line. The hypothetical pKDR/tKDR ratios of head and neck patient X and Y with different response to bevacizumab±erlotinib treatment are shown.

The response prediction curve generated for baseline pKDR/tKDR ratio can predict response to bevacizumab±erlotinib. For example, pKDR/tKDR ratios for hypothetical head and neck patients X and Y with PD and CR, respectively, predict a positive response with bevacizumab±erlotinib treatment.

### EXAMPLE 4

### PDX Index

We next sought to develop an algorithm that would allow us to mathematically combine the data from a variety of measured parameters to generate a number or value that would be predictive of patient outcome. Thus, the “PharmacoDynamic eXpression” (“PDX”) index or value was developed to assess treatment efficacy and quantitate probable response.

For drug-targeted therapy, the sum of % change of the tumor promoting factors (TPF), e.g., typically the drug targets and/or proliferation (ki67 positive), as well as % change of the tumor suppressing factor (TSF), e.g., cell death (TUNEL positive) and/or negative cell cycle regulators, before and after drug treatment is divided by the total number of TPF (n) and TSF factors (m) considered. Thus TPF1 is the % change of Promoting Factor 1 after treatment and TSF1 is the % change of Suppressing Factor 1 after treatment.

PDX score is generated by the formula:

PDX=[(100+TPF1)₁+(100+TPF2)₂+ . . . (100+TPFN)ₙ+(100−TSF1)₁+(100−TSF2)₂+ . . . (100−TSFM)ₘ]/(n+m)

wherein % decrease or % increase after drug treatment will render the values of TPF and TSF positive or negative, respectively. PDX score is between 0 and 100. In general, a lower PDX score predicts better clinical outcome resulting from a given treatment. A high PDX score indicates the treatment is not affecting the factors being monitored and a different treatment should be initiated.

For RTKIs designed to inhibit receptors X, Y, and Z, and induce cell death (TUNEL positive), pRTK and tRTK are measured by LSC-mediated quantification of X, Y, and Z before and after treatment. RTK generated by measuring pRTK_{X}/tRTK_{X} before and after treatment then calculating the % change for pRTK_{X}/tRTK_{X}. The PDX score is then generated by the following formula:

PDX=[(100+RTK_{X})+(100+RTK_{Y})+(100+RTK_{Z})+(100−TUNEL)]/(3+1)

wherein RTK is the % change for pRTK_{X}/tRTK_{X}, RTK is the % change for pRTK_{Y}/tRTK_{Y}, RTK_{Z} is the % change for pRTK_{Z}/tRTK_{Z}, and TUNEL is the % change in TUNEL intensity before and after treatment for the sample, 4 is the number of factors considered, and PDX is a score between 0-100.

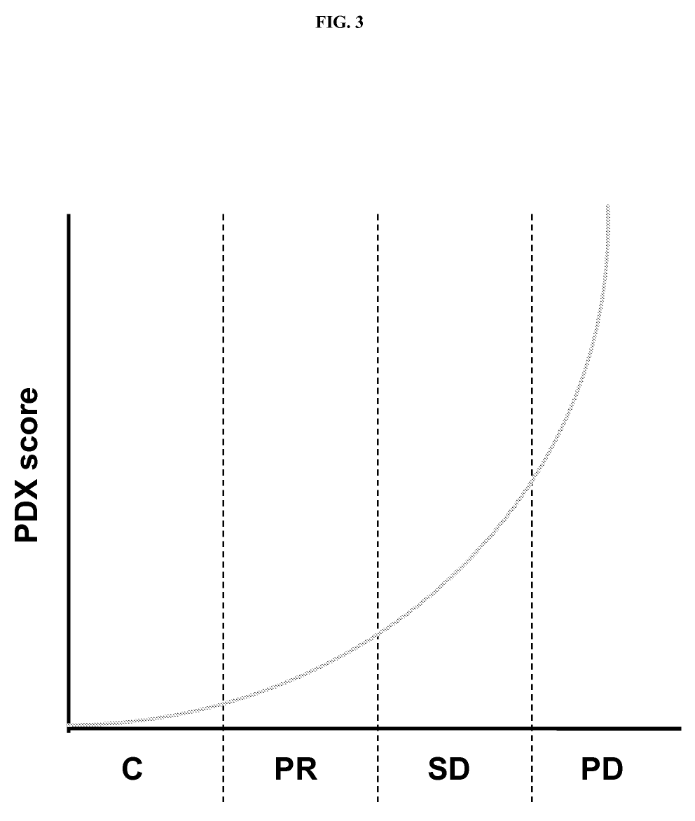

As described in FIG. 3, PDX index is a correlation between PDX and disease outcome (CR, PR, SD, and PD). In general, a lower PDX value predicts better treatment outcome, and vice versa.

pRTK/tRTK ratios are incorporated into PDX index ratings for each RTK and treatment options. Thus as pRTK/tRTK scores are assembled for patients with various cancer types, customized treatment regimes can be developed. Interactive databases score and compare pRTK/tRTK values for multiple RTKs across cancer types and with a variety of treatment regimes measuring pRTK/tRTK value before, during and after treatment. Other measured parameters can also be included in the PDX in the same way that TUNEL was included above. Treatment efficacy can then be predicted by PDX. Interactive data will identify drug resistance and determine the most effective treatment options. This will minimize use of ineffective compositions to which drug resistance has been developed and provide better treatment options.

Oncologists can predict a patient's response to RTKI treatment by comparing PDX values of a patient against a PDX index. The PDX index may be generated from a test population, using a disease model, or interactively based on patient population. PDX index may be selected based on cancer type, RTK measured, or treatment regimen. In general, the lower the PDX value against the PDX index predicts better treatment outcome, and vice versa. A hypothetical PDX index is illustrated in FIG. 3. Based on the PDX value, oncologists can optimize patient's treatment plan at the earliest possible stage in treatment. The formula is designed to incorporate molecular data with other parameters from imaging or pharmacokinetic information.

### REFERENCES

All references are listed herein for the convenience of the reader. Each is incorporated by reference in its entirety.

1. Gavrieli, Sherman, and Ben-Sasson, “Identification of programmed cell death in situ via specific labeling of nuclear DNA fragmentation.” J. Cell Biol. 119:493-501 (1992).

2. Hanks and Hunter, “Protein kinases 6. The eukaryotic protein kinase superfamily: kinase (catalytic) domain structure and classification.” FASEB J. 9:576-96 (1995).

3. Hubbard, “Structural analysis of receptor tyrosine kinases.” Prog Biophys Mol Bio1.71:343-58 (1999).

4. Hubbard and Till, “Protein tyrosine kinase structure and function.” Annu Rev Biochem.69:373-98 (2000).

5. Negoescu, et al., “TUNEL apoptotic cell detection in tissue sections: critical evaluation and improvement.” J. Histochem. Cytochem. 44:959-68 (1996).

6. Negoescu, et al., “In situ apoptotic cell labeling by the TUNEL method: improvement and evaluation on cell preparations.” F. Biomed. Pharmacother. 52:252-8 (1998).

What is claimed is:

1. A method of screening potential receptor tyrosine kinase inhibitors, said method comprising:
a) exposing a cell having a receptor tyrosine kinase (RTK) to a test agent;
b) measuring phosphorylation of said RTK in said cell to produce a pRTK amount,
c) measuring total RTK in said cell to produce a tRTK amount, and
d) determining a pRTK/tRTK ratio by dividing the pRTK amount by the tRTK amount,
wherein a dose dependent decrease in pRTK/tRTK ratio indicates that the test agent will have efficacy as an RTK inhibitor.

2. The method of claim 1, wherein said measuring phosphorylation of a receptor tyrosine kinase is by immunodetection and said measuring total receptor tyrosine kinase is by immunodetection.

3. The method of claim 1, wherein said pRTK/tRTK ratio is determined by simultaneously measuring pRTK and tRTK using flow cytometry, laser scanning cytometry, western blot, or dot blot.

4. The method of claim 1, wherein said test agent is selected from the group consisting of AEE788, SU11248, bevacizumab, erlotinib, cetuximab, trastuzumab, gefitinib, dasatinib, erlotinib, gefitinib, imatinib, lapatinib, nilotinib, sorafenib, sunitinib, vandetanib (ZD6474), and combinations thereof

5. The method of claim 1, wherein said test agent is selected from the group consisting of AEE788, SU11248, bevacizumab, erlotinib and combinations thereof

6. The method of claim 1, wherein said receptor tyrosine kinase is selected from the group consisting of epidermal growth factor receptor (EGFR), platelet derived growth factor receptor (PDGFR), vascular endothelial growth factor receptor (VEGFR), VEGFR2 (KDR), TIE receptor tyrosine kinase, protein tyrosine kinase (PYK), proto-oncogene tyrosine-protein kinase (c-Src), epithelial cell receptor protein-tyrosine kinases (EPH), human epidermal growth factor receptor (HER), stem cell tyrosine kinase receptor (KIT), platelet-derived growth factor receptor (PDGFR), and fms-related tyrosine kinase (FLT).

7. A method of assessing or monitoring the efficacy of a treatment comprising:
a) obtaining a cell having a receptor tyrosine kinase (RTK) from a patient having an RTK-mediated condition,
b) measuring phosphorylation of said RTK in said cell to produce a pRTK amount,
c) measuring total RTK in said cell to produce a tRTK amount,
d) determining a pRTK/tRTK ratio by dividing the pRTK amount by the tRTK amount,
wherein a low pRTK/tRTK ratio indicates that the treatment will have efficacy as an RTK inhibitor.

8. The method of claim 7, wherein said measuring phosphorylation of a receptor tyrosine kinase is by immunodetection and said measuring total receptor tyrosine kinase is by immunodetection.

9. The method of claim 7, wherein said pRTK/tRTK ratio is determined by simultaneously measuring pRTK and tRTK using flow cytometry, laser scanning cytometry, western blot, or dot blot.

10. The method of claim 7, wherein said treatment is selected from the group consisting of AEE788, SU11248, bevacizumab, erlotinib, cetuximab, trastuzumab, gefitinib, dasatinib, erlotinib, gefitinib, imatinib, lapatinib, nilotinib, sorafenib, sunitinib, vandetanib (ZD6474), and combinations thereof.

11. The method of claim 7, wherein said test agent is selected from the group consisting of AEE788, SU11248, bevacizumab, erlotinib and combinations thereof.

12. The method of claim 7, wherein said receptor tyrosine kinase is selected from the group consisting of epidermal growth factor receptor (EGFR), platelet derived growth factor receptor (PDGFR), vascular endothelial growth factor receptor (VEGFR), VEGFR2 (KDR), TIE receptor tyrosine kinase, protein tyrosine kinase (PYK), proto-oncogene tyrosine-protein kinase (c-Src), epithelial cell receptor protein-tyrosine kinases (EPH), human epidermal growth factor receptor (HER), stem cell tyrosine kinase receptor (KIT), platelet-derived growth factor receptor (PDGFR), and fms-related tyrosine kinase (FLT).

13. A method of screening a potential drug to treat a disease, said method comprising:
a) measuring one or more tumor suppressing factor (TSF) and one or more tumor promoting factor (TPF) values in a cell,
b) exposing said cell to a test agent with potential to treat a disease;
c) measuring said one or more tumor suppressing factor (TSF) and said one or more tumor promoting factor (TPF) values in said cell, and
d) calculating a PharmacoDynamic eXpression (PDX) value using the equation:
PDX=[(100+TPF1)₁+ . . . (100+TPFN)_{N}+(100−TSF1)₁+ . . . (100−TSFM)ₘ/(n+m)
wherein n is the number of TPF values and m is the number of TSF values, and
wherein a low PDX value indicates that the test agent will be effective to treat said disease.

14. The method of claim 13, wherein said one or more TPF values is measuring phosphor-receptor tyrosine kinase and total receptor tyrosine kinase ratio.

15. The method of claim 14, wherein said pRTK/tRTK ratio is determined by simultaneously measuring pRTK and tRTK using flow cytometry, laser scanning cytometry, western blot, or dot blot.

16. The method of claim 13, wherein said treatment is selected from the group consisting of AEE788, SU11248, bevacizumab, erlotinib, cetuximab, trastuzumab, gefitinib, dasatinib, erlotinib, gefitinib, imatinib, lapatinib, nilotinib, sorafenib, sunitinib, vandetanib (ZD6474), and combinations thereof.

17. The method of claim 13, wherein said test agent is selected from the group consisting of AEE788, SU11248, bevacizumab, erlotinib and combinations thereof

18. The method of claim 13, wherein said receptor tyrosine kinase is selected from the group consisting of epidermal growth factor receptor (EGFR), platelet derived growth factor receptor (PDGFR), vascular endothelial growth factor receptor (VEGFR), VEGFR2 (KDR), TIE receptor tyrosine kinase, protein tyrosine kinase (PYK), proto-oncogene tyrosine-protein kinase (c-Src), epithelial cell receptor protein-tyrosine kinases (EPH), human epidermal growth factor receptor (HER), stem cell tyrosine kinase receptor (KIT), platelet-derived growth factor receptor (PDGFR), and fms-related tyrosine kinase (FLT).

19. A database of cancer treatment profiles comprising:
a) phospho-receptor tyrosine kinase to total receptor tyrosine kinase (pRTK/tRTK) ratio for one or more receptor tyrosine kinase proteins, and
b) efficacy profile for one or more cancer treatments wherein said efficacy profile and pRTK/tRTK ratio are correlated.

20. The database of claim 19 wherein said receptor tyrosine kinase is selected from the group consisting of epidermal growth factor receptor (EGFR), platelet derived growth factor receptor (PDGFR), vascular endothelial growth factor receptor (VEGFR), VEGFR2 (KDR), TIE receptor tyrosine kinase, protein tyrosine kinase (PYK), proto-oncogene tyrosine-protein kinase (c-Src), epithelial cell receptor protein-tyrosine kinases (EPH), human epidermal growth factor receptor (HER), stem cell tyrosine kinase receptor (KIT), platelet-derived growth factor receptor (PDGFR), and fms-related tyrosine kinase (FLT).

21. The database of claim 19 wherein said treatment is selected from the group consisting of AEE788, SU11248, bevacizumab, erlotinib, cetuximab, trastuzumab, gefitinib, dasatinib, erlotinib, gefitinib, imatinib, lapatinib, nilotinib, sorafenib, sunitinib, vandetanib (ZD6474), and combinations thereof.

22. The method of claim 19, wherein said test agent is selected from the group consisting of AEE788, SU11248, bevacizumab, erlotinib and combinations thereof.

23. The database of claim 19 wherein said pRTK/tRTK ratio is determined using flow cytometry, laser scanning cytometry, western blot, or other immunodetection method.

24. The database of claim 19 wherein said pRTK/tRTK ratio is scored using a PDX index based on pRTK/tRTK ratio of one or more RTKs and efficacy of said cancer treatment.
